(19) United States
(12) Patent Application Publication
(10) Pub. No.: US 2022/0347347 A1
(43) Pub. Date: Nov. 3, 2022

(54) Ligament Repair Scaffold

(71) Applicant: Worcester Polytechnic Institute, Worcester, MA (US)

(72) Inventors: Carolyn Heighton, Worcester, MA (US); Logan Gaudette, Worcester, MA (US); Gregory Kaleshian, Worcester, MA (US); Camden Holm, Worcester, MA (US); Paul Bonarrigo, Worcester, MA (US); George Pins, Holden, MA (US); David Magit, Worcester, MA (US); Matthew Phaneuf, Ashland, MA (US); Nathaniel Long, Hudson, MA (US); Rayan Kassab, Taunton, MA (US)

(21) Appl. No.: 17/731,851

(22) Filed: Apr. 28, 2022

Related U.S. Application Data
(60) Provisional application No. 63/180,721, filed on Apr. 28, 2021.

(51) Int. Cl. A61L 27/38; A61L 27/54; A61L 27/56

(52) U.S. Cl. CPC A61L 27/386; A61L 27/54; A61L 27/56; A61L 2300/414; A61L 2430/10

(57) ABSTRACT

A ligament repair device and method is particularly suited to the demands of UCL repair using a scaffold device for surgical implantation across a torn ligament section. The implantable device includes an encapsulation of a therapeutic element for increasing cell proliferation and growth eluted through a controlled release storage element adapted to release the therapeutic agent over time as the cell growth progresses and the scaffold structure degrades or is absorbed in favor of the new cell growth for ligament repair. Reduced recovery time is afforded by the need for only an relatively small incision for implantation. The introduction of a physiologically relevant drug delivery scaffold encourages more rapid cell growth over passive, physical repairs to the connective tissue.

### RELATED APPLICATIONS

This patent application claims the benefit under 35 U.S.C. § 119(e) of U.S. Provisional Patent App. No. 63/180,721 filed Apr. 28, 2021, entitled “LIGAMENT REPAIR SCAFFOLD,” incorporated herein by reference in entirety.

### BACKGROUND

The ulnar collateral ligament (UCL) is a ligament that stabilizes the elbow. During throwing motions, the ligament experiences valgus loading that puts stress on the ligament. For athletes whose sports involve an overhead throwing motion, the UCL can experience a substantial magnitude of forces from the power generated by the throwing motion. This can lead to chronic or acute UCL injuries. The most detrimental injuries are grade II and III injuries which are partial and full tears of the ligament respectively. Grade II injuries can be treated using surgical or non-surgical methods. Non-surgical methods typically involve activity restrictions, orthotics, ice-compressions, anti-inflammatory medications, and physical therapy. These treatments require a substantial recovery period and still may not lead to complete healing or a return to previous level of play. Operative treatments such as a so-called “Tommy John” surgery are invasive with many risks and a long recovery period.

### SUMMARY

A ligament repair device and method is particularly suited to the demands of UCL repair using a scaffold device for surgical implantation across a torn ligament section. The implantable device includes an encapsulation of a therapeutic element for increasing cell proliferation and growth eluted through a controlled release storage element adapted to release the therapeutic agent over time as the cell growth progresses and the scaffold structure degrades or is absorbed in favor of the new cell growth for ligament repair. Reduced recovery time is afforded by the need for only a relatively small incision for implantation. The introduction of a physiologically relevant drug delivery scaffold encourages more rapid cell growth over passive, physical repairs to the connective tissue.

Configurations herein are based, in part, on the observation that baseball pitchers incur musculoskeletal movements often associated with UCL injuries and repair. Ulnar collateral ligament (UCL) injuries have been a growing epidemic in the baseball world. Many MLB (Major League Baseball) pitchers have undergone Tommy John surgery during their career. The UCL runs along the inner side of the elbow and attaches the humerus to the ulna. The ligament is responsible for providing stability in the elbow during overhead throwing motions. During such motions, the UCL undergoes valgus and varus loads which can cause injury to the ligament. These injuries can occur acutely or chronically overtime. It has been shown that over 50% of MLB players experience elbow pain from some form of strain to their UCL. The reason some players experience pain but do not require surgery is because there are varied grades of UCL tears.

Unfortunately, conventional approaches to UCL repair, such as Tommy John procedures, suffer from the shortcomings lengthy recovery periods and are also highly invasive. Accordingly, configurations herein substantially overcome the shortcomings of conventional approaches by providing an implantable scaffold applied via a single small (<10 cm, ideally around 4-6 cm) incision that is biocompatible and absorbable while delivering a therapeutic agent in a controlled release over time to facilitate cell regrowth in the surgical region.

The disclosed approach recognizes that overhead throwing athletes experience near failure loads to their ulnar collateral ligament (UCL) which often results in full or partial tears of the ligament. Conventional treatment methods reconstruct the existing anatomy with the use of an autograft tendon. In contrast, configurations herein disclose an implantable reparative device for partial UCL tears. In a particular configuration, an implantable scaffold made of lyophilized collagen and a silk hydrogel encapsulates and releases platelet derived growth factor-BB (PDGF-BB) over a two-week period via a scaffold arrangement encased in decellularized bovine dermis. In this manner, accelerated cell growth commences in conjunction with a scaffold for supplementing the strength of the healing ligament until sufficient healing occurs, and the scaffold degrades and becomes absorbed as the healed ligament regains load bearing ability. Other connective tissues in addition to the UCL can likewise benefit from the disclosed approach.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of an anatomical environment suitable for use with configurations herein;

FIG. 2 shows a ligament repair in the environment of FIG. 1 applied to a UCL structure;

FIGS. 3A-3D show surgical repair regions applicable to the approach of FIG. 2;

FIGS. 4A-4D show fabrication approaches for the implantable ligament repair device of FIG. 2;

FIG. 5 shows the average cell count between the triplicate wells for each concentration of PDGF-BB and the number of days between cell seeding and passaging;

FIG. 6 shows protein release at different time and test conditions; and

FIG. 7 is a force vs. displacement graph of tension in a decellularized bovine dermis.

### DETAILED DESCRIPTION

A UCL repair is presented below as an example surgical implantation of the ligament repair device. Alternate approaches with other connective tissues and various therapeutic agents and delivery mediums may also benefit from the disclosed approach.

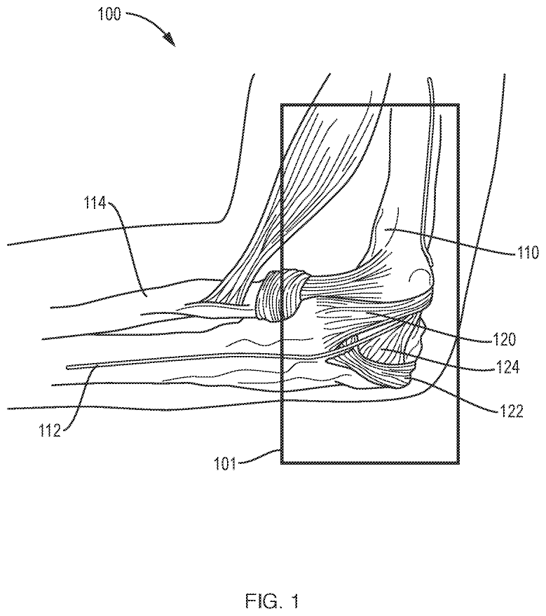

FIG. 1 is a context diagram of an anatomical environment suitable for use with configurations herein. In a patient anatomy 100, the ulnar collateral ligament (UCL) 101 is a ligament located in the elbow that runs from the humerus 110 to the ulna 112, adjacent the radius 114. This ligament complex is comprised of three bundles: the anterior 120, posterior 124, and transverse 122 bundle. The anterior bundle 120 of the UCL is the largest ligament and contains an anterior and posterior band. The anterior band is a single-layered ligament that runs organized longitudinal collagen fibers from the medial epicondyle to the sublime tubercle. This ligament activates during extension (30-90 degrees). The posterior band is a thicker ligament comprised of less organized collagen fibers that attaches from the humeral epicondyle to the medial ulna and is activated in flexion (90-120 degrees) of the elbow. The ligament complex works to provide stability to the elbow during valgus and varus loading with most of the load being placed on the anterior bundle 120, hence often being the focal point of failure such as tears.

In basic anatomical function, blood flows into the UCL from the superior ulnar collateral artery. This artery stems from the brachial artery of the medial upper arm and runs through the elbow joint. Smaller arteries such as the superior ulnar collateral artery and the anterior ulnar recurrent run behind the elbow. These arteries provide the ligament with nutrients and growth factors that facilitate the maintenance and regeneration of the UCL. A study that researched the vascularity of the ligament showed that there was a denser blood supply at the proximal end of the ligament compared to the distal end. According to the study, the proximal end of the UCL was shown to have 68% vascular penetration compared to 39% at the distal end. The difference in blood flow along the UCL directly effects the healing process. Configurations herein leverage bloodflow in conjunction with accelerated cell growth provided by the therapeutic agent.

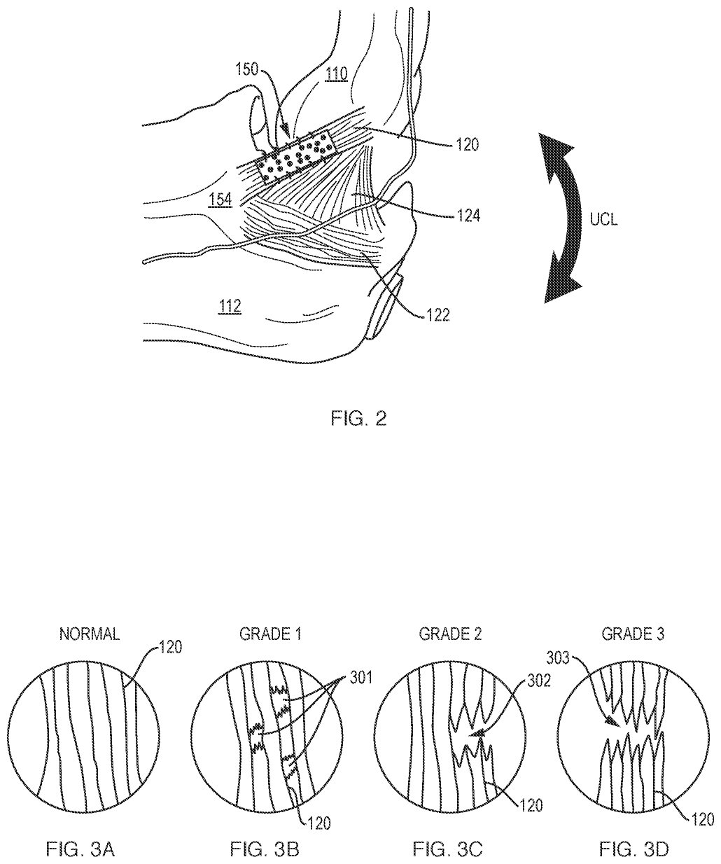

FIG. 2 shows a ligament repair in the environment of FIG. 1 applied to the UCL structure to address a tear in the anterior bundle 120. An implantable repair device 150 (device) surgically attaches to a torn ligament, secured by sutures 152 and containing impregnated and/or encapsulated, time-release therapeutic agent 154. In the examples below, collagen sponges are of interest for their demonstrated ability to provide mechanical stability to hydrogel scaffolds. Silk hydrogels can encapsulate therapeutic agents and release them over time. Growth factors, such as platelet-derived growth factor-BB (PDGF-BB), are involved in the natural ligament healing process and can be used to aid in recovery. The symbiotic combination develops a physiologically relevant drug delivery scaffold to serve as an implantable reparative treatment system for the UCL.

Platelet-derived growth factor (PDGF) is one of many growth factors that regulate cell growth and division. In particular, PDGF enhances blood vessel formation, the growth of blood vessels from already-existing blood vessel tissue, mitogenesis, i.e. proliferation, of mesenchymal cells such as fibroblasts, osteoblasts, tenocytes, vascular smooth muscle cells and mesenchymal stem cells as well as chemotaxis, the directed migration, of mesenchymal cells. Platelet-derived growth factor is a dimeric glycoprotein that can be composed of two A subunits (PDGF-AA), two B subunits (PDGF-BB), or one of each (PDGF-AB).

FIGS. 3A-3D show surgical repair regions applicable to the approach of FIG. 2. Referring to FIGS. 3A-3D. FIG. 3A shows a normal, healthy ligament such as the anterior bundle 120, with no compromise or injury. FIG. 3B shows a grade 1 tear; a grade I tear may indicate strain, stretching, and small tears or perforations 301 throughout the UCL. In FIG. 3C, a grade II tear indicates a larger tear 302 across most of the UCL partially compromising the ligaments structural integrity. Lastly, a grade III tear is a complete rupture 303 of the UCL, which is characterized by a loss of function of the arm, shown in FIG. 3D. The grade II tear, where the ligament is not completely severed but rather retains a portion of continuity, is particularly amenable to the scaffold and healing regrowth as provided by the repair implant device herein.

Grade II tears can be difficult to heal because they typically do not require total reconstructive surgery. However, they are extremely difficult to heal utilizing exclusively noninvasive methods such as physical therapy. Physical therapy can work in some cases, but it cannot heal a large percentage of grade II tears enough to return the patient to at least a pre-injury level of function. UCL reconstruction is extremely invasive and undesirable for those who want to return quickly to their previous level of activity.

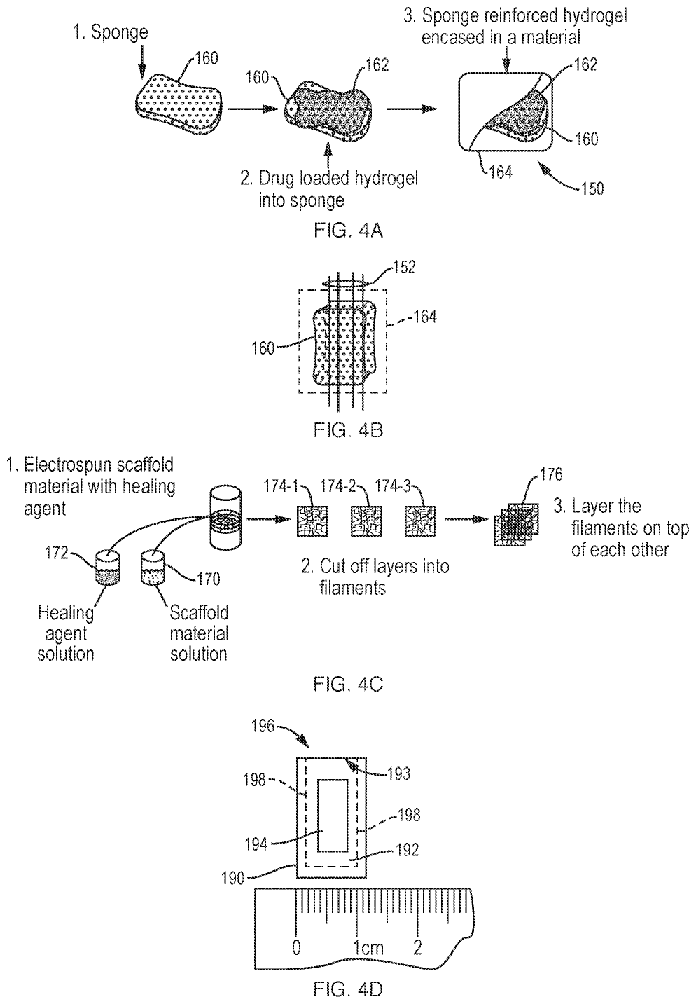

FIGS. 4A-4D show fabrication approaches for the implantable ligament repair device of FIG. 2. Referring to FIGS. 2-4A, the ligament repair implant device includes a structural scaffold adapted to engage torn tissue. In FIG. 4A, the structural scaffold is a collagen sponge 160. A drug 162 including a therapeutic agent for increasing cell proliferation is loaded into the scaffold. The loaded, implanted drug also includes a controlled release storage element adapted to release or elute the therapeutic agent over time. The controlled release storage element releases the therapeutic agent based on an absorbance longevity of the structural scaffold, so that the scaffold persists a sufficient time to allow cell regrowth to progress to the point where the support of the scaffold is no longer needed. A casing 164 encapsulates the structural scaffold and is adapted for biocompatible attachment to a tissue repair region including the surgical site of the anatomy region 100. FIG. 4B shows attachment of the loaded sponge 160′ and encapsulation 164 via sutures 152.

Certain properties are beneficial for proper function of a biological scaffold. In addition to being biocompatible, most biological scaffolds should be bioabsorbable so as to not negatively affect the long-term function of the ligament or tendon. Biological scaffolds should degrade at a similar rate to the rate at which the body naturally produces new tissue in the location of scaffold implantation. This will protect the cells that grow into the scaffold from the environment while they are healing, but then allow the cells to be exposed to the environment once they have matured. If the scaffold degrades at the same rate as tissue ingrowth it will also help to prevent the stress shielding in the tendon or ligament

In the example of FIGS. 4A-4B, the controlled release storage element further includes a hydrogel encapsulating the therapeutic agent, such that the controlled release storage element is a silk hydrogel loaded with a therapeutic agent including PDGF-BB. This provides at least 2 weeks of release to promote cell growth and about a 4-6 week longevity of the collagen scaffold. The result is a supportive scaffold that can be fixed in place on the UCL by current surgical techniques and will aid in healing by providing a slow release of growth factors or other biological additives.

FIG. 4C shows an alternate approach comingling the healing agent and scaffold material as layered filaments. An electrospun scaffold material solution 170 and a therapeutic agent in a solution 172 are prepared. Filaments from the solutions are alternated and interwoven into a series of layers 174-1 . . . 174-3, and layered together as an implantable scaffold 176.

FIG. 4D shows an alternate arrangement of a scaffold 190 is composed of two sheets of electrospun polylactic-glycolic acid (PLGA) 192 that were electronically welded together to create a pocket 196. The controlled release storage element includes an electrospun scaffold material with a therapeutic healing agent formed into filaments layered in an interspersed arrangement. The pocket was filled with a strip of zein 194 in order to allow for a more controlled drug release from the scaffold in vivo. The center of the pocket was then filled with a PRP (platelet rich plasma) hydrogel. The scaffold 190 would be filled through a preinserted tube which could accept a needle, and then be closed either using biostaples or some other form of medical adhesive.

Specifications and dimensions are as follows. The scaffold, shown in FIG. 4D, is formed from two electrospun PLGA sheets 192. These sheets will be 2.0×1.0 cm each and will be ultrasonically welded together to form the seams 198. Zein, a 20 kDa structural protein present in maize endosperm cells, was procured. A Zein solution was prepared in an organic alcohol solvent and electrospun onto a 167 mm diameter mandrel at a 15 cm gap distance and +21 kV for 300 minutes. Material thickness ranged between 250-350 um. Fiber diameters ranged between 700-900 nm. The scaffold was tack welded across the entire perimeter at 1.0 mm from the edge of the scaffold leaving an open slot 193 at the top. In this slot, an electrospun Zein segment 194 1.5×0.5 cm will be inserted into the slot and embedded inside the scaffold. This inner material will contain the growth factors, such as PRP and TGF-β, that are intended for the therapeutic effect of the scaffold. The slot end of the scaffold will be sealed using ultrasonic and tack welding in accordance with the rest of the scaffold. The therapeutic agent, PRP, will be inserted by puncturing the scaffold and administering the PRP through an 18-gauge needle. The hole created by the needle will be sealed using a biologically compatible glue.

PLGA is a biocompatible synthetic polymer in the family of polyesters. PLGA is made by the combination of glycolic and lactic acid. This material is affordable, easy to work with as it can be molded or used in 3D printing applications. PLGA is a biodegradable material whose degradation rate and crystallinity is controlled by the ratio of glycolic and lactic acid. The main drawbacks of this material are the acidic degradation products. The acidic byproducts produced by the degradation of the material can have adverse effects on surrounding tissue.

Platelet rich plasma (PRP) consists of high concentrations of growth factors in a small amount of plasma. It is believed that the use of PRP will encourage healing of the ligament through increased cell growth and blood vessel development.

Zein has gained significant traction in its use for biomedical applications. Zein based composites have been studied for tissue and bone regeneration, drug delivery, and wound healing. Zein is a beneficial material for the body as is has been found to be both biocompatible and biodegradable with its main limitation being its mechanical strength. Zein is a renewable natural source, as it is derived from corn, making it economically feasible as well. Zein itself is a major storage protein located in corn endosperm. It can be extracted and processed in numerous different ways such as mechanical elongation, antisolvent precipitation and electrospinning. Each processing technique has very different outcomes, giving zein a wide range of applications.

Transforming growth factor (TGF) is believed to be active in nearly all phases of tendon healing with an added importance in the initial inflammatory phase. TGFB has a wide variety of effects on tendon and ligament healing such as stimulation of cell migration, collagen production, regulation of proteinases, termination of cell proliferation and guiding fibronectin binding. Studies have shown an increase of cell proliferation and collagen production after the introduction of TGFB.

As indicated above, experimental results for PDGF-BB and silk hydrogel indicated symbiotic results. The complete growth factor eluding structure of the ligament repair implant device 150 contains three components: a collagen sponge scaffold, a drug loaded silk hydrogel, and a casing made of decellularized bovine dermis, referring again to FIG. 4A. This whole scaffold device design would then be sutured to a partially torn UCL or other amenable connective tissue.

In various configurations, the therapeutic agent may include one or more of mesenchymal stem cells, bone marrow, platelet-like particles, platelet rich plasma (PRP) and isolated growth factors. The therapeutic agent may include growth factors such as platelet derived growth factors (PDGF), transforming growth factor B (TGFB), insulin-like growth factors I and II (IGF), vascular endothelial growth factors (VEGF) and fibroblast growth factor (FGF). The controlled release storage element may be formed using one or more of collagen sponges, keratin films, chitosan sponges, gelatin, silk hydrogels and electrospun zein mats.

A general process for forming the ligament repair scaffold using these components includes generating a solution of a biocompatible, controlled release storage element, and adding a therapeutic agent to the controlled release storage element, to form a pregel solution, such that the therapeutic agent is selected based on an ability to increase cell proliferation. A portion of the pregel solution is dispensed into a repository having a shape based on a surgical repair region, and a correspondingly shaped scaffold element deposited in the repository for accepting the pregel solution for subsequent elution and release. To ensure uniform distribution, a second portion of the pregel solution is then dispensed into the repository, and the scaffold element removed for eluting the therapeutic agent into the surgical repair region following implantation.

To form a particular example configuration, polydimethylsiloxane (PDMS) molds were employed. A silk fibroin solution was sonicated at 5 wt % in 1.5 mL batches for 90 seconds. Then, we added 1.00 μg/mL of PDGF-BB to the silk pregel solution. Using a positive displacement pipet, we injected 150 μL of pregel into the PDMS mold, placed a collagen sponge on top, and then injected another 150 μL of pregel solution into the mold. We placed the collagen sponge reinforced hydrogels into a vacuum chamber for 20 minutes to release air bubbles. The scaffolds sat at 20° C. in a dark box for 48 hours to gel. The dimensions of the final hydrogel were 20 mm×7.5 mm×2 mm and the dimensions of the dermis were 24 mm×10 mm×0.4 mm.

Validation through cell proliferation testing sought to characterize the growth factor's efficacy in increasing cell proliferation, and therefore ligament reparation. We cultured 3T3 mouse fibroblast cells in 10% fetal bovine serum culture media at 99% humidity, 37° C. and 5% CO₂ and at concentrations of 0, 0.167, 0.417, 0.667, or 1.00 μg/mL of PDGF-BB in triplicates. We seeded cells at an initial concentration of 30,000 cells/1 mL of media in a 12-well plate and passaged them on days three and five. Once the concentration of PDGF-BB that showed the highest rate of proliferation had been determined, it was loaded into the final silk gels and the viability of PDGF-BB eluding from the collagen-infused silk hydrogel scaffold was determined.

Drug elution testing was performed to characterize the release profile of albumin from sponge reinforced hydrogels to ensure that enough therapeutic agent was loaded and released over a two-week period. Bovine serum albumin (BSA) served as a model protein for this testing. We loaded BSA into three different scaffold conditions: silk hydrogels (SA), collagen sponge reinforced hydrogels (SAC), and collagen sponges with no albumin (C). We suspended five scaffolds of each condition in 1 mL of DPBS (−), and supernatant samples of 1 mL were taken at 0, 1, 2, 3, 4, 7, 10 and 14 days. Finally, we conducted a BCA (bicinchoninic acid) assay to quantify a cumulative drug release profile. We then performed the same protocol with PDGF-BB instead of BSA and measured the concentration of eluded PDGF-BB with a human PDGF-BB ELISA Kit.

We calculated the diffusion coefficient of PDGF-BB through diffusivity testing of the scaffold casing to ensure it would help extend the release profile of PDGF-BB. We utilized an Ussing chamber to test the mass transfer of 2 mg/mL BSA in DPBS (−) from one enclosed chamber through decellularized bovine dermis to an acceptor chamber of DPBS (−) without BSA. Every 24 hours for 7 days, we collected 100 μL samples from the acceptor chamber and measured protein concentration with a BCA Assay. We determined the diffusion coefficient of BSA in DPBS (−) across decellularized dermis in cm²/second from the measured concentrations using Equations A and B where Nis the total solute mass in system (6.8 mg), Vis the total volume, C₂ is the protein concentration in the acceptor chamber at a given time in mg/mL, h is thickness of the barrier (0.40 mm), t is time in days, A is surface area of the barrier (0.709 cm²), and τ is the scaling factor.

$$D = - ln ( 1 - ( \frac{{C}_{2} ( t )}{( \frac{N}{V} )} ) ) \cdot h \cdot \frac{τ}{t} \qquad ( A )$$

$$τ = \frac{( {V}_{1} + \frac{A h}{2} ) ( {V}_{2} + \frac{A h}{2} )}{AV} \qquad ( B )$$

We mechanically tested the scaffold casing to determine its material properties and ensure it would withstand surgical manipulation, requiring a stiffness between 3 and 17 N/mm and a failure load greater than 13 N. The tensile testing protocol was performed using an E1000 Instron with a strain rate of 25%/minute. As the decellularized bovine dermis is anisotropic, five samples were tested in one direction (D1) and then rotated 90 degrees to test it in the perpendicular direction (D2).

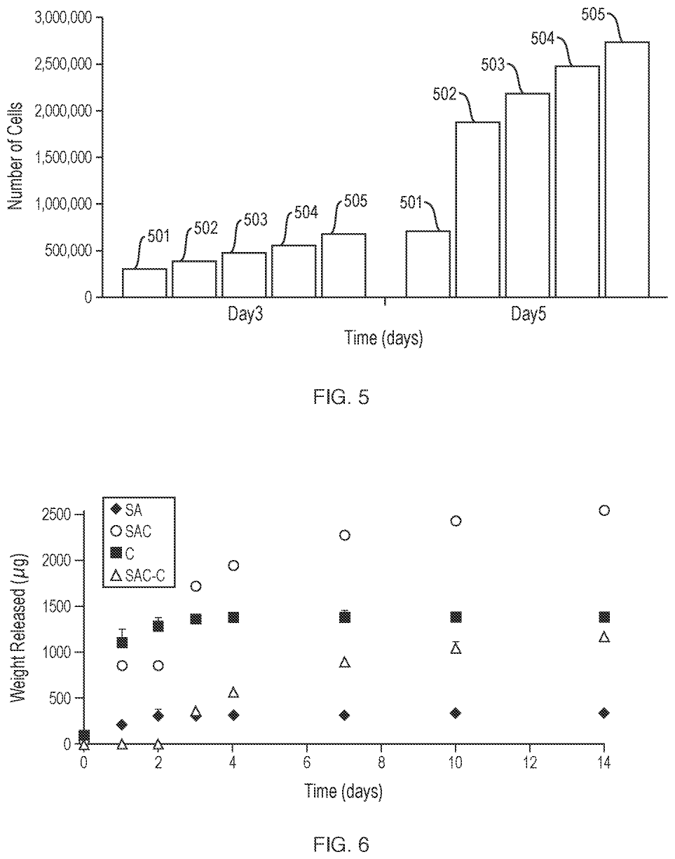

After we passaged cells on days three and five, the concentration of PDGF-BB that resulted in the highest cell proliferation was determined. FIG. 5 shows the average cell count between the triplicate wells for each concentration of PDGF-BB and the number of days between cell seeding and passaging. Referring to FIG. 5, higher concentrations of PDGF-BB led to an increased cell count, although cells were 100% confluent on day five for all concentrations besides the control. The histogram in FIG. 5 depicts Number of Cells Counted when Exposed to Different Concentrations of PDGF-BB, where the concentration increments are (in ug/ml) 0.0 (501); 0.167 (502); 0.417 (503); 0.667 (504) and 1.00 (505).

FIG. 6 shows protein release at different time and test conditions, showing cumulative release of BSA from Various Scaffold Designs. Referring to FIG. 6, since the BCA assay can read the collagen protein, the eluded of amount of protein from C was subtracted from the SAC scaffold (represented in triangles). From this data, the general trends show that SAC scaffolds sustained a more controlled release than the SA scaffolds, which had a large burst release. This indicates that collagen is advantageous in lengthening the release period of albumin.

The effect of the casing was also evaluated. It was determined the diffusion coefficient of BSA through the decellularized dermis to be 9.07×10⁻⁸±7.38 cm²/s (n=7). This value is comparable to one study that found that the diffusion coefficient of BSA across a human cornea membrane to be 3.1±1.0×10⁻⁸ cm²/s. [6] The BSA concentrations in the acceptor chamber on days 0, 1, 2, 3, 4, 5, 6, 7 were 0.00 mg/mL, 0.00 mg/mL, 0.0385 mg/mL, 0.0498 mg/mL, 0.0966 mg/mL, 0.150 mg/mL, 0.182 mg/mL, 0.190 mg/mL respectively. By day 7, 8.28% of the BSA had diffused through the decellularized bovine dermis, supporting the hypothesis that the dermis casing would slow down the release of PDGF-BB to the injury site.

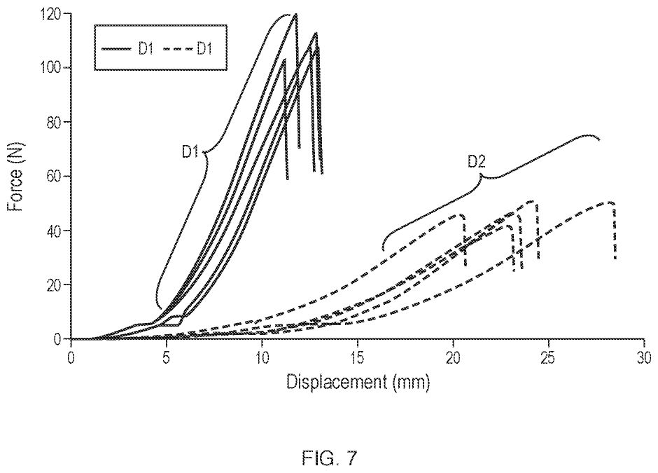

The decellularized bovine dermis in its first direction had an average stiffness of 18.33±1.35 N/mm and failure load of 109.91+/−6.24 N, while the second direction of the decellularized bovine dermis had an average stiffness of 4.59±0.63 N/mm and failure load of 45.05±5.83 N. The force-displacement curves from these results are shown in FIG. 7, depicting a force vs. displacement graph of decellularized bovine dermis tested in tension in a first direction (D1) and second direction (D2). Referring to FIG. 7, it was determined that the design would be mechanically uncompromised during surgical manipulation, as the decellularized dermis fell within predetermined stiffness and failure load constraints.

The results showed that the collagen sponge reinforced silk albumin hydrogel sustained a more controlled release compared to the silk albumin hydrogel alone. We found that the concentration of PDGF-BB that promoted the highest cell proliferation was 1.00 μg/mL, which was statistically significant (p=0.00015) when compared to the control. To extend the release period of PDGF-BB to 14 days and provide the proper mechanical properties, the scaffold should be encased in decellularized bovine dermis. Preliminary testing showed that the decellularized bovine dermis casing should slowdown the release of PDGF-BB from the sponge reinforced hydrogel scaffold, as less than 10% of BSA diffused the dermis after 7 days.

The release of PDGF-BB over two weeks is critical to the reparation of the UCL. With this new device, it is anticipated that the patients' native ligament may heal much quicker due to the presence of PDGF-BB. In addition, the risk of post-operative injuries will be reduced. Future recommendations for this technology are to test crosslinked collagen, to experiment with different concentrations of silk hydrogels to improve the release profile and to perform studies in small and large animal models. With the successful completion of these trials, the device could begin to be tested for the treatment of UCL tears in overhead throwing athletes, as well as other high stress/load musculoskeletal contexts.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A ligament repair implant device, comprising:
a structural scaffold adapted to engage torn tissue;
a therapeutic agent for increasing cell proliferation; and
a controlled release storage element adapted to release the therapeutic agent over time.

2. The device of claim 1 further comprising a casing encapsulating the structural scaffold and adapted for biocompatible attachment to a tissue repair region.

3. The device of claim 1 wherein the controlled release storage element elutes the therapeutic agent.

4. The device of claim 1 wherein the controlled release storage element releases the therapeutic agent based on an absorbance longevity of the structural scaffold.

5. The device of claim 1 wherein the controlled release storage element further comprises a hydrogel encapsulating the therapeutic agent.

6. The device of claim 1 wherein the structural scaffold is a collagen sponge, and the controlled release storage element is a silk hydrogel loaded with platelet derived growth factor-BB (PDGF-BB).

7. The device of claim 1 wherein the controlled release storage element includes an electrospun scaffold material with a therapeutic healing agent formed into filaments layered in an interspersed arrangement.

8. The device of claim 1 wherein the therapeutic agent includes one or more of mesenchymal stem cells, bone marrow, platelet-like particles, platelet rich plasma (PRP) and isolated growth factors.

9. The device of claim 1 wherein the therapeutic agent includes growth factors selected from the group consisting of platelet derived growth factors (PDGF), transforming growth factor B (TGFB), insulin-like growth factors I and II (IGF), vascular endothelial growth factors (VEGF) and fibroblast growth factor (FGF).

10. The device of claim 1 wherein the controlled release storage element includes one or more of collagen sponges, keratin films, chitosan sponges, gelatin, silk hydrogels and electrospun zein mats.

11. A method for forming a ligament repair scaffold, comprising:
generating a solution of a biocompatible, controlled release storage element
adding a therapeutic agent to the controlled release storage element, to form a pregel solution, the therapeutic agent selected based on an ability to increase cell proliferation;
dispensing a portion of the pregel solution into a repository, the repository having a shape based on a surgical repair region;
adding a scaffold element to the repository for accepting the pregel solution;
dispensing a second portion of the pregel solution into the repository; and
removing the scaffold element for eluting the therapeutic agent into the surgical repair region following implantation.

12. The method of claim 11 further comprising dispensing a first portion in a quantity of half the amount of therapeutic agent for release, and the second portion defining the remaining amount of therapeutic agent for release.

13. The method of claim 11 wherein the scaffold element includes a collagen sponge.

14. The method of claim 11 wherein the pregel solution is formed from a silk hydrogel solution.

15. The method of claim 11 wherein the shape of the repository and a dimension of the scaffold element are based on a UCL repair region.

16. A ligament repair scaffold device, comprising:
opposed electrospun sheets welded at perimeter seams to form a scaffold enclosure having an open slot;
an electrospun Zein segment embedded between the opposed electrospun sheets via insertion through the open slot; and
a therapeutic agent in the scaffold enclosure resulting from needle punctures through the scaffold enclosure.

17. The device of claim 16 wherein the Zein segment is based on a 40% (w:v) Zein solution and growth factors.

18. The device of claim 1 wherein the therapeutic agent is PRP.
